(19) United States
(12) Patent Application Publication
(10) Pub. No.: US 2021/0220314 A1
(43) Pub. Date: Jul. 22, 2021

(54) AMINO ACID COMPOSITION FOR ENABLING FULFILMENT OF THE AMINO ACID REQUIREMENTS OF A MONOGASTRIC ANIMAL SUCH AS A HUMAN OR A PIG

(71) Applicant: VETAGRO INTERNATIONAL S.R.L., Reggio Emilia (IT)

(72) Inventor: Andrea PIVA, Reggio Emilia (IT)

(21) Appl. No.: 17/265,769

(22) PCT Filed: Jul. 31, 2019
(86) PCT No.: PCT/IB2019/056528

§ 371 Date: Feb. 3, 2021

(30) Foreign Application Priority Data
Aug. 7, 2018 (IT) 102018000007947

(51) Int. Cl. A61K 31/198; A61K 31/405; A61K 31/401; A61K 38/17; A61K 35/741; A61K 45/06; A61K 33/26; A61K 33/04; A61K 33/06; A61K 33/00; A61K 33/30; A61K 33/34; A61K 47/44; A61K 47/02; A61K 9/00; A23L 33/175; A23L 33/19; A23L 33/115; A23L 33/15; A23L 33/16; A23L 33/135; A23L 33/00; A23K 20/147; A23K 20/158; A23K 20/174; A23K 20/22; A23K 20/24; A23K 20/20; A23K 20/28; A23K 50/30; A23K 10/18

(52) U.S. Cl. CPC A61K 31/198; A23V 2002/00; A61K 31/401; A61K 38/1709; A61K 35/741; A61K 45/06; A61K 33/26; A61K 33/04; A61K 33/06; A61K 33/00; A61K 33/30; A61K 33/34; A61K 47/44; A61K 47/02; A61K 9/0056; A23L 33/175; A23L 33/19; A23L 33/115; A23L 33/15; A23L 33/16; A23L 33/135; A23L 33/40; A23K 20/147; A23K 20/158; A23K 20/174; A23K 20/22; A23K 20/24; A23K 20/30; A23K 20/28; A23K 50/30; A23K 10/18; A61K 31/405

(57) ABSTRACT

The present invention relates to a composition comprising at least one amino acid and/or at least one whey protein and a controlled-release lipid matrix. Furthermore, the present invention relates to said composition for use in a method of treatment with a supply of amino acids or of protein deficiency and of a pathology, symptom and/or disorder deriving from said protein deficiency, in a monogastric subject, preferably a human subject or a pig.

The present invention relates to a method for preparing a composition comprising at least one amino acid, preferably at least two amino acids, and salts thereof. Furthermore, the present invention also relates to a composition for human or animal use, preferably for monogastric animals, comprising at least one amino acid and/or at least one whey protein and a controlled-release lipid matrix, wherein said composition can be obtained by means of said method. Furthermore, the present invention also relates to said composition for human or animal use, preferably for monogastric animals, in a method of therapeutic and non-therapeutic treatment with a supply of amino acids or of a protein deficiency and a pathology, symptom and/or disorder deriving from said protein deficiency.

The development and maintenance of skeletal muscle mass are determined by the sum of the processes of muscle protein synthesis (abbreviated MPS, process at the basis of hypertrophy) and muscle protein breakdown (abbreviated MPB, process at the basis of atrophy). The preservation and development of muscle mass in humans, determined by the homeostatic equilibrium between MPS and MPB, are essential elements for maintaining metabolic health and independent locomotion, or in general a better quality of life ([1] Dideriksen et al. 2013). This equilibrium between MPS and MPB can be disturbed by various factors, including some chronic diseases, lack of use of muscles and aging. The loss of muscle mass and muscular strength (sarcopenia), in fact, is among the main causes of increased mortality and morbidity and a reduced quality of life in the elderly ([2] Nowson and O'connell 2015). It has been verified that the intravenous administration of amino acids (AAs) in volunteer subjects, by promoting hyperaminoacidaemia and hyperinsulinaemia, stimulates MPS ([3] Philips, 2014). However, muscle protein synthesis is a process considered to be “saturable”, so the amino acid composition of the protein source and the amount of essential amino acids (EAAs) taken in through the diet prove to be crucial. In humans, there are nine EAAs, i.e. AAs that the body is not capable of synthesising de novo and must thus be acquired through the diet: histidine, isoleucine, leucine, lysine, methionine, phenylalanine, threonine, tryptophan and valine ([4] DRI, 2006). As regards pigs, to this list of EAAs we should add: arginine, cysteine and tyrosine. Of the nine EAAs in humans, recent studies have demonstrated that leucine, one of the branched-chain AAs (BCAAs), plays a crucial role in MPS by activating signalling cascades in the pathway of the molecule mTORC1, both in humans and in pigs. This AA, in fact, has been identified as the principal anabolic signal among the different AAs (Bohè et al. 2003). During the postprandial phase (1-4 h after a meal) MPS is high, resulting in a positive muscle protein balance, whereas the rate of MPS is lower in the fasting phase and the protein balance is negative ([5] Jager et al. 2017). Furthermore, it has been demonstrated that the blood concentration of EAAs regulates the rate of protein synthesis in muscles at rest and after exercise ([5] Jager et al. 2017).

To date, the commercially available compositions (pharmaceutical compositions, dietary supplements, foods, feeds, feed additives, nutraceutical compositions) for human or animal protein supplementation do not make reference to particular AAs and do not take into consideration the specificity of each subject, understood as the actual qualitative/quantitative requirements. In fact, in order to maintain a suitable equilibrium between MPS and MPB, it is important to take into consideration that the protein and individual amino acid (AA) requirements differ from subject to subject in relation to various factors, first and foremost age ([4] DRI 2006). For example, in an elderly subject (over 75 years old) there is a so-called “anabolic resistance”, with the occurrence of imbalances in the mTORC1 pathway ([6] Mitchell et al. 2016), and the protein intake declines, an aspect that the daily doses recommended with reference to a subject's protein requirements do not take into consideration ([2] Nowson and O'Connell 2015). Another influencing factor is the degree of sedentariness and, in the case of a sporty individual, the type of exercise (aerobic or resistance). Exercise, in fact, particularly resistance exercise, is one of the drivers of MPS, together with diet ([7] Stokes et al. 2018); thus, the amino acid requirements of sporty individuals are different. There are also differences between males and females. In the literature, the majority of studies aimed at identifying amino acid requirements have been conducted mainly on male subjects, with the exception of a few studies in which it was demonstrated that amino acid requirements vary based on the phase of the menstrual cycle ([8] Elango et al. 2008). Furthermore, such requirements vary under exceptional conditions such as pregnancy and breastfeeding ([4] DRI, 2006). Furthermore, it is well known that there is a difference in the prevalence of the type of muscle fibres depending on race and, despite there not being any studies aimed at investigating the AA composition based on racial genetics, it is highly probable that AA requirements vary in relation to the prevalent type of muscle fibres.

These differences show that it is necessary to define a supplementation of specific AAs which is targeted at the different real needs of each subject and takes account of all the above-described parameters. Furthermore, it would be very useful to be able to ensure a larger amount and constancy of amino acids in the blood, thereby enabling a more constant blood bioavailability of amino acids over the 24 hours by limiting the fluctuations between the main meals.

The literature has highlighted that there are various problems in the administration of amino acids to human subjects or monogastric animals. In particular, free amino acids are highly acidic; therefore, when they are administered enterally they can cause problems by inducing heartburn or stomach ulcers. Furthermore, tryptophan degrades at an acidic pH such as that in the stomach (pH 2-3), in particular during fasting. Thus, there is a high demand for amino acids in protected forms that permit their transit in the stomach without either causing damage to the walls of the gastric tract or undergoing degradation.

The technical problem that the present invention addresses is therefore to provide a valid solution enabling the development and preparation of compositions (pharmaceutical compositions, dietary supplements, foods, feeds, feed additives or nutraceutical compositions) suitable for providing a specific supply of amino acids, from both a qualitative and quantitative standpoint, to a human or animal subject which is suited to the specific needs of said subject (e.g. age, gender, genetics of race or breed/type of muscle fibres, state of health, physical activity engaged in, specific blood values). In order to overcome said technical problem, the present invention provides a method making it possible to prepare a composition comprising amino acids that are ad hoc-selected, in terms of quality and quantity, for each subject or group of subjects based on physical conditions and the type of needs of the subject or group of subjects, as detailed below.

Furthermore, the present invention provides a composition obtained by means of the method of the invention which is free of side effects, easy to prepare and cost-effective.

In addition, the technical problem that the present invention addresses and solves is to provide compositions suitable for providing a supply of amino acids and/or proteins to a monogastric subject, preferably a human subject or a pig, to support the normal development of muscle mass or to favour the increase thereof.

In addition, the technical problem that the present invention addresses and solves is to provide said subject with amino acids and/or proteins in such a way that the blood bioavailability thereof is constant over a period of 2 to 24 hours, in order to limit the fluctuations in the blood levels thereof between the main meals.

Finally, the technical problem that the present invention addresses and solves is to provide amino acids and/or gastro-protected proteins that can be administered enterally without causing damage to the walls of the gastric tract and/or without undergoing degradation in the gastric tract.

These objects and still others, which will become clear from the detailed description that follows, are achieved by the method and the composition of the present invention thanks to the technical features claimed in the appended claims.

The Applicant, following an intense phase of research and development, has found a method for determining, based on several parameters of a subject, or group of subjects, how to select the amino acids (quality and quantity) to be formulated in order to provide a composition that can meet the needs for an adequate supply of amino acids for said subject.

The subject matter of the present invention relates to a method (briefly, method of the invention) for preparing a composition (briefly, composition of the invention for human or animal use, preferably for monogastric animals) comprising at least one amino acid, preferably at least two amino acids, or acceptable pharmaceutical or food grade salts thereof;

said method comprising the steps of:
evaluating and/or quantifying, for a human subject or an animal, preferably a monogastric animal, at least a first parameter selected from the group comprising or, alternatively, consisting of: gender, age, race or breed or amino acid composition of the muscle fibre based on race or breed, type of sports activity engaged in, type of work performed, daily diet or a set of two or more of said first parameters; and/or
evaluating and/or quantifying, for a human subject or an animal, preferably a monogastric animal, at least a second parameter selected from the group comprising or, alternatively, consisting of: plasma nitrogen concentration, blood creatinine, blood creatine phosphokinase, blood lactic acid or lactate after physical exertion, number of daily steps, or saliva sample or parameters obtained from a genetic characterisation or a set of said second parameters; followed by,
selecting, on the basis of said at least a first parameter or set of said first parameters and on the basis of said at least a second parameter or set of said second parameters, at least one amino acid, preferably at least two amino acids; followed by
selecting, on the basis of said at least a first parameter or set of said first parameters and on the basis of said at least a second parameter or set of said second parameters, a first amount of said at least one amino acid, preferably of said at least two amino acids; followed by,
formulating said at least one amino acid, preferably said at least two amino acids, selected and quantified in the preceding steps in a composition suitable for being administered to said subject or animal.

The method of the invention can envisage that only said at least a first parameter or set of said first parameters will be evaluated; or that only said at least a second parameter or set of said second parameters will be evaluated. Preferably, the method of the invention envisages that both said at least a first parameter or set of said first parameters will be evaluated and said at least a second parameter or set of said second parameters will be evaluated.

The evaluation (or measurement) of said second parameters is performed according to standard methods known to the person skilled in the art.

Creatinine is a protein that is mostly found in muscles. An increase in said protein, after ruling out kidney damage with tests, can depend on intense physical activity.

Creatine phosphokinase (CPK) intervenes in the energy mechanism associated with creatine, it is present in muscles (MM type), in the heart (MB) and in the brain (BB). It is generally released into the blood by muscles (skeletal and heart) when there is muscle fibre damage or fatigue: intense exertion, a sprain or injury, surgical interventions or, in the most severe cases, myocardial infarction, neuromuscular diseases or thyroid abnormalities.

Lactic acid or lactate is a by-product of lactacid anaerobic metabolism. It is a compound that is toxic for cells, whose accumulation in the bloodstream is correlated to the manifestation of so-called muscle fatigue.

Advantageously, the amino acids are thus selected and quantified on the basis of a study of real individual requirements of a human subject or an animal, preferably a monogastric animal, obtained by studying the AA omposition of muscle fibre and/or analysis of saliva samples or blood samples or blood tests of the subject or animal. In a preferred embodiment, said composition may be determined by collecting a saliva sample to collect DNA of the single individual and/or determining the presence of genes involved in the anabolic process and/or correlatable to the composition of the muscle fibre and/or correlatable to specific pathologies by carrying out standard methods known to the person skilled in the art. The study of the real protein requirements on the basis of said first and/or second parameters can be conducted using different methods, including the nitrogen balance or indicator amino acid oxidation (IAAO) method, or that of measuring the oxidation of individual essential AAs ([9] LARN 2017). The latter two methods are the ones considered most appropriate for defining more specific AA requirements.

In a preferred embodiment, the steps of evaluating said at least a first parameter and/or said at least a second parameter are further followed, in addition to the steps of selecting at least one amino acid, preferably at least two amino acids, and the amount thereof for the purpose of the present invention, by the steps of:
selecting, on the basis of said at least a first parameter or set of said first parameters and/or on the basis of said at least a second parameter or set of said second parameters, at least one non-amino acid ingredient selected from among one or more organic or inorganic acids, one or more aromatic components, at least one vitamin, at least one mineral salt, at least one antioxidant, at least one probiotic bacterial strain, or a set of said non-amino acid ingredients; followed by:
selecting, on the basis of said at least a first parameter or set of said first parameters and/or on the basis of said at least a second parameter or set of said second parameters, a second amount of said at least one non-amino acid ingredient or of said set of said non-amino acid ingredients;
formulating said at least one non-amino acid ingredient or set of said non-amino acid ingredients in said second amount together with said at least two amino acids in said composition.

As described above, in the context of the present invention, the term “non-amino acid ingredient” means a compound of a non-amino acid nature selected in group C comprising or, alternatively, consisting of: organic or inorganic acids, aromatic components, vitamins, mineral salts, antioxidants, probiotic bacterial strains, prebiotics and enzymes.

Advantageously, said vitamin is a vitamin of the A, B, C, D, and/or K groups; preferably a B group vitamin selected in the group comprising or, alternatively, consisting of B1, B2, B3, B4, B5, B6, B7, B8, B9, B10, B11, B12 and mixtures thereof.

Advantageously, said one mineral salt is an organic or inorganic salt of a cation of a metal, such as, for example Fe, Se, Mg, Ca, K, Zn or Cu.

Advantageously, said one antioxidant is selected from among N-acetyl cysteine (NAC), Coenzyme Q10 (CoQ10), L-acetylcarnitine, and the like.

The subject matter of the invention further relates to a composition, preferably obtained by means of the method of the invention, comprising:
(i) a mixture (briefly, mixture of the invention or mixture of the active components) which comprises or, alternatively, consists of (a) at least one amino acid, preferably at least two amino acids, or acceptable pharmaceutical or food grade salts thereof, and/or (b) a whey protein
   and, optionally, said composition comprises
(ii) at least one acceptable pharmaceutical or food grade additive and/or excipient.

Furthermore, the composition of the invention comprises (iii) a lipid matrix as described below, optionally comprising coating additives (iii.1).

In one embodiment, said at least one amino acid or said at least two amino acids comprised in the composition of the invention, which comprises said (i) mixture and (iii) lipid matrix and, optionally, (ii) additive and/or (iii.1) coating additive (as defined below), are principally amino acids that are essential for monogastric subjects, such as humans or pigs; the composition of the invention preferably comprises two or more essential amino acids, for example three, four or five. Said (a) at least one essential or non-essential amino acid is selected in group A comprising or, alternatively, consisting of: histidine, isoleucine, leucine, lysine, methionine, phenylalanine, threonine, tryptophan, valine, arginine, cysteine, tryptophan, glutamine and mixtures thereof; preferably glutamine, phenylalanine, lysine, methionine, threonine, tryptophan, valine, isoleucine, histidine or leucine; more preferably leucine, valine and histidine; even more preferably leucine.

In one embodiment, said group A does not comprise lysine and/or tryptophan.

In one embodiment, said group A does not comprise tryptophan when the composition of the invention comprises sulfamethazine or sulfadimidine (SMT) (IUPAC name 4-amino-N-(4,6-dimethylpyrimidine-2-yl) benzenesulfonamide, CAS no. 57-68-1).

In one embodiment, said group A does not comprise tryptophan when the (iii) lipid matrix of the invention comprises or, alternatively, consists of long-chain fatty acids, preferably a mixture of stearic acid, palmitic acid, oleic acid and myristic acid.

In one embodiment of the composition of the invention comprising said (i) mixture and (iii) lipid matrix and, optionally, (ii) additive and/or (iii.1) coating additive, said (a) at least one amino acid is a mixture of amino acids selected from the group B of mixtures comprising or, alternatively, consisting of:
(B.1) leucine, valine and isoleucine (BCAA);
(B.2) leucine and at least one or more amino acids selected from group A, preferably one or more amino acids selected from among lysine, methionine, threonine, tryptophan, valine, isoleucine, histidine and glutamine, such as, for example, the mixtures: leucine and lysine; leucine and methionine; leucine and threonine; leucine and tryptophan; leucine and valine; leucine and isoleucine; leucine and histidine; leucine and glutamine; leucine and lysine and one selected from among methionine, threonine, tryptophan, valine, isoleucine, histidine and glutamine; leucine and methionine and one selected from among lysine, threonine, tryptophan, valine, isoleucine, histidine and glutamine; leucine and threonine and one selected from among lysine, methionine, tryptophan, valine, isoleucine, histidine and glutamine; leucine and tryptophan and one selected from among lysine, methionine, threonine, valine, isoleucine, histidine and glutamine; leucine and valine and one selected from among lysine, methionine, threonine, tryptophan, isoleucine, histidine and glutamine; leucine and isoleucine and one selected from among lysine, methionine, threonine, tryptophan, valine, histidine and glutamine; leucine and histidine and one selected from among lysine, methionine, threonine, tryptophan, valine, isoleucine and glutamine; leucine and glutamine and one selected from among lysine, methionine, threonine, tryptophan, valine, isoleucine and histidine; leucine and isoleucine and valine and one selected from among lysine, methionine, threonine, tryptophan, histidine and glutamine;
(B.3) leucine, isoleucine, valine, lysine, methionine, threonine and tryptophan;
(B.4) leucine, isoleucine, valine, lysine, methionine, threonine, tryptophan and histidine;
(B.5) lysine, methionine, threonine, tryptophan;
(B.6) lysine, methionine, threonine, tryptophan and valine.

When said subject is a human subject, said mixture of amino acids selected from said group B is preferably selected from among (B.1), (B.2), (B.3) and (B.4).

When said subject is a pig, said mixture of amino acids selected from said group B is preferably selected between (B.5) and (B.6).

Preferably, in said binary mixtures of group B, such as, for example, those of group (B.1): leucine and lysine, leucine and methionine, leucine and threonine, leucine and tryptophan, leucine and valine, leucine and isoleucine, leucine and histidine, leucine and glutamine, the two amino acids are preferably in a ratio to each other by weight comprised in the range 1:10 to 10:1, preferably 1:5 to 5:1, more preferably 1:3 to 3:1, even more preferably 1:1.

In the context of the present invention, the term “amino acids” relates to L-α-amino acids, i.e. those whose amino group and carboxyl group are bonded to the same carbon atom, called, precisely, α-carbon, in an L configuration, and are thus endowed with a respective optical activity, with the sole exception of glycine, which is achiral. Amino acids are the constituent units of proteins (proteinogenic); depending on the type, number and sequential order in which the different amino acids bind it is possible to obtain an enormous number of proteins. In nature, we classically know 20 proteinogenic amino acids. Our body is able to synthesise some of the amino acids necessary to build proteins, but is not capable of constructing others, which are therefore defined “essential amino acids” (EAAs) and must be introduced through foods.

“Whey protein” or whey proteins is a mixture of proteins isolated from the whey of cow's milk, the liquid matter that constitutes a by-product of cheesemaking. The proteins in cow's milk consist of about 20% whey protein and 80% casein protein, whereas the protein in human milk consists of 60% whey and 40% casein. Whey proteins are in general a mixture of β-lactoglobulins, α-lactalbumins, serum albumins, and other minor fractions, which are soluble in their native form, independent of the pH. The protein fraction in whey (about 10% of the dry matter within the whey) comprises four main protein fractions and six minor protein fractions. The main protein fractions of whey are: β-lactoglobulins (˜65%), α-lactalbumins (˜25%), and serum albumins (˜8%); whilst the minor fractions (˜2%) of whey are: lactoferrins, immunoglobulins, glycomacropeptides, lactoperoxidase and lysozyme. Furthermore, whey proteins consist of 40-50% essential amino acids (EAAs) and are considered a rich source of these amino acids.

The amino acid composition of the whey proteins, as reported in Gorissen et al. (Amino Acids, 50:1685-1695, 2018), is shown below.

TABLE 1

| | g/100 g of |
|---|---|
| Essential AAs (EAAs) | whey proteins |
| Threonine | 5.4 |
| Methionine | 1.8 |
| Phenylalanine | 2.5 |
| Histidine | 1.4 |
| Lysine | 7.1 |
| Valine | 3.5 |
| Isoleucine | 3.8 |
| Leucine | 8.6 |
| Non-Essential AAs | |
| (NEAAs) | |
| Serine | 4.0 |
| Glycine | 1.5 |
| Glutamic acid | 16.0 |
| Proline | 8.7 |
| Cysteine | 0.1 |
| Alanine | 2.6 |
| Tyrosine | 4.4 |
| Arginine | 2.9 |

In one embodiment of the composition of the invention comprising said (i), (iii) and optionally (ii), said (a) at least one amino acid, or said at least two amino acids, and/or said (b) whey protein are present overall (only (a) or only (b) or (a) and (b)) in the composition of the invention at a concentration (%) by weight comprised in the range 1% to 90% relative to the total weight of the composition, preferably 10% to 50%, even more preferably 15% to 45%.

The composition of the invention further comprises, in addition to the (i) mixture and, optionally, to (ii) additives and/or excipients, (iii) a coating matrix (or controlled-release lipid coating matrix or controlled-release lipid matrix or lipid matrix of the invention), wherein said (iii) coating matrix embeds or incorporates or disperses and/or microencapsulates said (i) mixture, according to what is defined in the present invention and according to the process of preparation of the present invention.

Summing up, the composition of the invention comprises:
(i) a mixture which comprises or, alternatively, consists of (a) at least one or a mixture of amino acids selected from said group A or group B of mixtures, or acceptable pharmaceutical or food grade salts thereof, and/or (b) a whey protein;
(iii) a controlled-release lipid matrix as defined in the context of the present invention, wherein said (iii) lipid matrix embeds or incorporates or disperses said (i) mixture, according to what is defined in the present invention and according to the process of preparation of the invention;
   and, optionally, said (iii) lipid matrix comprises said (iii.1) at least one coating additive (as described below);
   and, optionally, said composition comprises
(ii) at least one acceptable pharmaceutical or food grade additive and/or excipient.

In one embodiment, the composition of the invention, comprising said (i) and (iii) and, optionally, (ii) and/or (iii.1), comprises, in addition to (a) at least one amino acid, preferably at least two amino acids, and/or one (b) said whey protein, also (c) at least one non-amino acid ingredient selected in group C, as defined above, comprising or, alternatively, consisting of organic or inorganic acids, aromatic components, vitamins, mineral salts, antioxidants, probiotic bacterial strains, prebiotics and enzymes.

In the presence of said (iii) coating matrix, said (c) non-amino acid ingredient can be comprised in the (i) mixture and can itself also be microencapsulated or embedded or incorporated or dispersed by/in said (iii) lipid coating matrix or, alternatively, it can be comprised in the composition but not comprised in the (i) mixture microencapsulated or embedded by the (iii) coating matrix. If present, said (c) non-amino acid ingredient selected in group C is preferably comprised in the (i) mixture and is thus itself also embedded or incorporated or dispersed by/in said (iii) lipid matrix.

In a preferred embodiment, the composition of the invention comprises:
said (i) mixture which comprises or, alternatively, consists of (a) at least one or a mixture of amino acids selected from said group A or group B, or salts thereof, and/or (b) a whey protein;
said (iii) controlled-release lipid matrix (and optionally (iii.1)); and
(c) at least one vitamin selected from among vitamins of the A, B, C, D, E or K groups; preferably a B group vitamin selected in the group comprising or, alternatively, consisting of B1, B2, B3, B4, B5, B6, B7, B8, B9, B10, B11, B12 and mixtures thereof.

The composition of the invention preferably comprises:
said (a) at least one or a mixture of amino acids, or salts thereof, selected from said group A or group B (such as B.1, B.2, B.3, B.4, B.5 or B.6), preferably leucine, leucine and lysine, leucine and methionine, leucine and threonine, leucine and tryptophan, leucine and valine, leucine and isoleucine, leucine and histidine, leucine and glutamine or a leucine, isoleucine and valine mixture;
said (iii) controlled-release lipid matrix and optionally (iii.1)); and
a B group vitamin selected in the group comprising or, alternatively, consisting of B1, B2, B3, B4, B5, B6, B7, B8, B9, B10, B11, B12 and mixtures thereof. Advantageously, in this embodiment, said (a) at least one or a mixture of amino acids, or salts thereof, and said B group vitamin are in ratio by weight ((a):vitamin B) comprised in the range 1:10 to 10:1, preferably 1:5 to 5:1, more preferably 1:3 to 3:1, even more preferably 1:1.

Said (iii) coating matrix enables a controlled release of the amino acid after administration to a subject (in short, controlled-release coating matrix).

Said (iii) coating matrix which enables a controlled release of the amino acid or mixture of amino acids and/or of the whey protein after administration to a subject comprises or, alternatively, consists of:
at least one saturated or unsaturated, free or esterified fatty acid, having a number of carbon atoms comprised in the range C10-C30, preferably C14-C24, and/or
at least one triglyceride having chains of saturated or unsaturated fatty acids, having a carbon number comprised in the range C6-C30, preferably C14-C24, and/or
at least one or a mixture of waxes having a number of carbon atoms comprised in the range C16-C36, preferably C24-C36; hereinafter called, for the sake of simplicity, controlled-release lipid coating matrix; and, optionally,
at least one coating additive (iii.1) as described below.

Said (iii) controlled-release lipid coating matrix is capable of releasing the components present in the (i) mixture (i.e. (a) and/or (b) and, optionally (c)) as a function of time and, if administered enterally, as a function of the digestive process. Advantageously, said (iii) controlled-release lipid coating matrix is capable of ensuring a greater amount and constancy of amino acids in the blood and enables a more constant blood bioavailability of the amino acids and/or whey protein and/or non-amino acid components (c) over the 24 (or 18) hour period, advantageously limiting the fluctuations in the same between the main meals.

The (i) mixture that is microencapsulated or embedded or incorporated or dispersed with/in said (iii) controlled-release lipid coating matrix is produced by means of the production method described in the patent document EP 1391155 A1 in paragraphs [0048]-[0049] and [0077] (in short, process of preparation of the invention); said paragraphs are incorporated by reference in the present description. In short, said process of preparation of the invention comprises the steps of:
step (I), preparing said (iii) controlled-release lipid matrix according to any one of the embodiments of the invention and, if present, said (ii) at least one additive and/or excipient so as to obtain a homogeneous mass (I) (temperature around 80° C.-120° C.), followed by
step (II), dispersing said (i) mixture of active components (i.e. (a) and/or (b) and, optionally (c)), according to any one of the embodiments of the invention, in said homogeneous mass (I) so as to yield a mass (II) (temperature around 55° C.-70° C., followed by
step (III), spraying the mass (II) in a cold room (temperature below 15° C.) so as to yield the composition of the invention, preferably in the form of relatively spherical particles, wherein the active components comprised in said (i) mixture (i.e. (a) and/or (b) and, optionally (c)) and, if present, said (ii) additive are dispersed or incorporated or embedded by/in said (iii) controlled-release lipid matrix.

In other words, the composition of the invention obtained from the process of preparation of the invention is an aggregate of (a) and/or (b) and, if present, (c) and, optionally, (ii) dispersed in said (iii) controlled-release lipid matrix.

Consequently, in the context of the present invention, the expression “(iii) a coating matrix, wherein said (iii) coating matrix embeds or incorporates or disperses and/or microencapsulates said (i) mixture” means an aggregate of the components comprised in the (i) mixture, namely, (a) and/or (b) and, if present, (c), dispersed in (iii) the controlled-release lipid matrix.

The expression “(iii) a coating matrix, wherein said (iii) coating matrix embeds or incorporates or disperses and/or microencapsulates said (i) mixture” does not identify components comprised in the (i) mixture, namely, (a) and/or (b) and, if present, (c), coated with a film of said (iii) controlled-release lipid matrix.

Furthermore, the expression “(iii) a coating matrix, wherein said (iii) coating matrix embeds or incorporates or disperses and/or microencapsulates said (i) mixture” does not identify components comprised in the (i) mixture, namely, (a) and/or (b) and, if present, (c), in the form of tablets, pills or like forms coated with the (iii) controlled-release lipid matrix or with film of (iii).

The advantages of the composition of the invention, in particular the long-term (2 hours-24 hours) blood bioavailability of the active components comprised in the mixture or composition of the invention, derive both from the physicochemical properties of said (iii) controlled-release lipid matrix and from the particular process of preparation of the invention which enables the active components of the (i) mixture (i.e. (a) and/or (b) and, if present, (c)) to be incorporated or dispersed or embedded in the (iii) lipid matrix.

In fact, said (iii) controlled-release lipid matrix, in addition to providing a controlled release of the active components comprised in the (i) mixture (i.e. (a) and/or (b) and, if present, (c)) in the gut also favours the gastroprotection thereof, since said (iii) matrix is stable at the acidic pH of the stomach (pH 2-3).

Consequently, said (iii) lipid matrix, by incorporating and/or embedding said active components (i.e. (a) and/or (b) and, if present, (c)) enables the transit thereof through the stomach without undergoing degradation and without the amino acids, acidic substances, causing damage to the walls of the gastric tract. When the composition of the invention reaches the gut, where the pH has a higher value than in the stomach (pH 6-7.5), said (iii) lipid matrix dissolves slowly, allowing a controlled release of the active components and, therefore, a constant blood bioavailability in an range of time comprised from 2 hours to 24 hours. Furthermore, the gut has an enzymatic endowment rich in lipases, which, by digesting the lipid matrix, enable the controlled release of the active components.

For the purpose of demonstrating the effectiveness of the lipid matrix in the gastroprotection of the active components comprised in a composition, a study was performed in which the presence of sorbic acid and vanillin (markers) was monitored in the content of different sections of the gastrointestinal tract of a first group of pigs to which a composition comprising natural acids, including sorbic acid, and flavourings, including vanillin, and encapsulated in a lipid matrix had been administered orally and a second group of pigs to which the same components in free form (not encapsulated in the lipid matrix) had been administered. Said study reveals that the two markers, sorbic acid and vanillin, are present in the different intestinal sections only when administered in encapsulated form in the lipid matrix, since the lipid matrix enables the stomach to be bypassed and allows a slow release at the intestinal level, where the markers are absorbed and made available in the blood, with a consequent increase of blood bioavailability.

Furthermore, a study was performed in order to demonstrate the prolonging of the bioavailability of the active ingredients over time following the encapsulation thereof with a lipid matrix, using sulfamethazine as the study marker. In particular, a composition comprising sulfamethazine encapsulated with a lipid matrix was administered orally to a first group of pigs and a composition comprising sulfamethazine in free form (not encapsulated in a lipid matrix) was administered to a second group at the dose of 1 g/pig. Eight hours after administration, the absorbed fraction of the sulfamethazine incorporated in the lipid matrix showed to be 31.8±13% smaller than the sulfamethazine in free form. With the form encapsulated in the lipid matrix, the absorption of sulfamethazine was completed in 24 hours, whereas with the free form it was completed in 10 hours, thus revealing the effect of the time-controlled release and of constant blood bioavailability over 24 hours for the form encapsulated with the lipid matrix.

Sorbic acid, vanillin and sulfamethazine were used instead of amino acids as markers of the release from a lipid matrix because analytically it is nearly impossible to determine the presence, at the intestinal level, of the limiting amino acids released by the compositions undergoing analysis considering the high content of dietary amino acids, cells from intestinal desquamation and microbial proliferation.

It follows that the addition of suitable amino acids and/or proteins to the diet of monogastric subjects, preferably human subjects or pigs, via the composition of the invention, makes it possible to increase the efficiency of the amino acids/proteins administered and, consequently, both to reduce the excess of undigested proteins in the gut of the subject, thus limiting the onset of potential infectious pathologies, and to reduce the nitrogen excreted by said subject, thus limiting the environmental impact produced by livestock.

Furthermore, satisfying the requirements of limiting amino acids or proteins by means of the composition of the invention helps to decrease the percentage of proteins in the diet of the monogastric subject, bringing about an economic advantage in terms of the cost of feeds when said subject is an animal, for example a pig.

Furthermore, the compositions of the invention are free of side effects and can thus be administered to a wide range of animal or human subjects, also including paediatric subjects, the elderly and pregnant women.

Finally, the compositions of the invention are easy to prepare and cost-effective.

In the context of the present invention, the term “subject” means a human subject or a monogastric animal, preferably a human subject or a pig.

In the context of the present invention, the term “monogastric” means an animal whose stomach has a single chamber in which chemical and enzymatic digestion take place. In contrast, polygastric animals or ruminants: have a stomach made up of four different chambers: rumen, reticulum, omasum and abomasum (which is the equivalent of the stomach of monogastric animals since it is the only one endowed with gastric mucosa). Belonging to this group are the Camelids (endowed with a three-chambered stomach) and the Ruminants in the strict sense (Bovids, Cervids, Giraffids, etc.). Polygastric animals have a better ability to digest plant feedstuffs thanks to rumination and microbial digestion, which takes place in the rumen.

“Triglycerides” (or triacylglycerols) are neutral esters of glycerol, in which the chains of three long-chain fatty acids are present in place of the hydrogen atoms of the hydroxyl groups. The length of the fatty acid chains in the common structures of triglycerides can be from 5 to 28 carbon atoms, but 17 and 19 are most common.

The term “fatty acids” (abbreviated FAs) means aliphatic monocarboxylic acids which are prevalently, but not exclusively, long-chain with an even number of carbon atoms, unbranched and acyclic (i.e. consisting of molecules that do not have chains forming a closed ring). Fatty acids can be saturated (if their molecule has only C—C single bonds) or unsaturated (if they have C═C double bonds).

The term “waxes” means long-chain fatty acid esters with high molecular weight monohydric alcohols. Waxes can be of vegetable origin or animal origin (beeswax). Beeswax is made up of different compounds, including, for example: hydrocarbons 14%, monoesters 35%, diesters 14%, triesters 3%, hydroxy monoesters 4%, hydroxy polyesters 8%, acid esters 1%, acid polyesters 2%, free acids 12%, free alcohols 1%, unidentified 6%. The main components of beeswax are palmitates, palmitic acid, hydroxy palmitates and oleate esters formed by long chains (30-32 carbon atoms) of aliphatic alcohols, with a 6:1 ratio between the two main components, triacontyl palmitate (myricyl palmitate) CH₃(CH₂)₂₉O—CO—(CH₂)₁₄CH₃ and cerotic acid CH₃(CH₂)₂₄COOH. Beeswax has a melting point comprised between 62° C. and 64° C. The density at 15° C. ranges between 0.958 and 0.970 g/cm³. Beeswax can be classified into two large categories: European type and Oriental type. The saponification number is 3-5 for the European type and 8-9 for the Oriental type.

Advantageously, said fatty acid comprised in the (iii) controlled-release lipid coating matrix can be a hydrogenated or non-hydrogenated fatty acid of vegetable and/or animal origin.

Advantageously, said triglyceride comprised in the (iii) controlled-release lipid coating matrix can be a hydrogenated or non-hydrogenated triglyceride of vegetable and/or animal origin.

Advantageously, said waxes comprised in the (iii) controlled-release lipid coating matrix can be of vegetable and/or animal origin; preferably beeswax.

In a preferred embodiment, said (iii) controlled-release lipid coating matrix comprising or, alternatively, consisting of at least one hydrogenated fatty acid of vegetable and/or animal origin and/or at least one hydrogenated triglyceride of vegetable and/or animal origin and/or at least one wax, and, optionally, at least one of said coating additives (iii.1); preferably at least one hydrogenated fatty acid of vegetable origin and/or at least one hydrogenated triglyceride of vegetable origin and/or at least one wax of animal origin.

The hydrogenated vegetable triglycerides are selected from the group comprising: palm oil, sunflower oil, corn oil, rapeseed oil, peanut oil, olive oil and soybean oil.

The triglycerides of animal origin, preferably hydrogenated, are selected from among: chicken fat, hydrogenated chicken fat, beef tallow and pork lard.

The composition can preferably comprise said (iii) coating matrix (controlled-release lipid coating matrix) in the various embodiments thereof in an amount (%) by weight comprised in the range 10% to 80% relative to the total weight of the composition; preferably 40% to 60%, more preferably 45% to 55%.

In one embodiment of the invention, the composition of the invention, comprising said (i) and (iii) and, optionally, (ii) and/or (iii.1), comprises said (i) mixture, which comprises (a) and/or (b) and, optionally (c) according to any one of the embodiments of the invention, in a % by weight comprised in the range 1% to 90% relative to the total weight of the composition, preferably 10% to 50%, more preferably 15% to 45%, and said (iii) controlled-release lipid matrix, according to any one of the embodiments of the invention, in a by weight comprised in the range 10% to 80% relative to the total weight of the composition; preferably 40% to 60%, more preferably 45% to 55%. Said % of (iii) represents the total % of (iii), independently of the components comprised in (iii), for example comprising or not comprising (iii.1).

In a preferred embodiment, the composition of the invention comprises:
(i) a mixture which comprises or, alternatively, consists of leucine, or a salt thereof and, optionally, one or more amino acids selected from said group A or group B;
(iii) a controlled-release lipid matrix as defined in the context of the present invention which comprises or, alternatively, consists of at least one fatty acid and/or triglyceride and/or waxes or mixtures thereof, preferably selected from among: palm oil, sunflower oil, corn oil, rapeseed oil, peanut oil, soybean oil, olive oil, beeswax and mixtures thereof, wherein said lipid matrix embeds or incorporates or disperses the active components of the (i) mixture, providing gastroprotection to the active components of the (i) mixture, a controlled release thereof in the gut and a constant blood bioavailability thereof over a period comprised from 2 to 24 hours; and, optionally, said matrix comprises (iii.1); and, optionally, said composition comprises (ii); wherein said (i) and (iii) are present in a % by weight as defined in the present invention.

In a preferred embodiment, the composition of the invention comprises:
(i) a mixture which comprises or, alternatively, consists of a mixture of leucine and isoleucine and valine, or salts thereof, and, optionally, one or more amino acids selected from said group A or group B;
(iii) a controlled-release lipid matrix as defined in the context of the present invention which comprises or, alternatively, consists of at least one fatty acid and/or triglyceride and/or waxes or mixtures thereof, preferably selected from among: palm oil, sunflower oil, corn oil, rapeseed oil, peanut oil, soybean oil, olive oil, beeswax and mixtures thereof, wherein said coating matrix embeds or incorporates or disperses the active components of the (i) mixture, providing gastroprotection to the active components of the (i) mixture, a controlled release thereof in the gut and a constant blood bioavailability thereof over a period comprised from 2 to 24 hours; and, optionally, said matrix comprises (iii.1); and, optionally, said composition comprises (ii); wherein said (i) and (iii) are present in a % by weight as defined in the present invention.

In a preferred embodiment, the composition of the invention comprises:
(i) a mixture which comprises or, alternatively, consists of (b) a whey protein and, optionally, one or more amino acids selected from said group A or group B;
(iii) a controlled-release lipid coating matrix as defined in the context of the present invention which comprises or, alternatively, consists of at least one fatty acid and/or triglyceride and/or waxes or mixtures thereof, preferably selected from among: palm oil, sunflower oil, corn oil, rapeseed oil, peanut oil, soybean oil, olive oil, beeswax and mixtures thereof, wherein said coating matrix embeds or incorporates or disperses the active components of the (i) mixture, providing gastroprotection to the active components of the (i) mixture, a controlled release thereof in the gut and a constant blood bioavailability thereof over a period comprised from 2 to 24 hours; and, optionally, said matrix comprises (iii.1);
   and, optionally, said composition comprises (ii); wherein (i) and (iii) are present in a % by weight as defined in the present invention.

The (i) mixture microencapsulated or embedded or incorporated or dispersed in/with said (iii) coating matrix (controlled-release lipid coating matrix) in the various embodiments thereof forms, by means of the process of preparation of the invention, spherical particles having a particle diameter (average particle diameter) comprised in the range 100 μm to 2000 μm, preferably 200 μm to 1500 μm, more preferably 250 μm to 1000 μm. In particular, when the composition of the invention is intended for human subjects, said average particle diameter is preferably comprised in the range 100 μm to 1000 μm. When the composition of the invention is instead intended for pigs, said average particle diameter is preferably comprised in the range 500 μm to 2000 μm, more preferably 500 μm to 1500 μm or 500 μm to 1000 μm.

The (iii) coating matrix of the invention (controlled-release lipid coating matrix), in the various embodiments thereof illustrated above, can further comprise one or more coating additives (iii.1). The coating additives (iii.1) are selected from the group comprising or, alternatively, consisting of: fumed silica, calcium stearate, magnesium stearate, calcium sulphate, precipitated silica, calcium silicate, aluminium silicate and hydrophobic silica. The coating additives (iii.1) used serve to increase the viscosity of the matrix and reduce the permeability thereof. The (iii) coating matrix of the invention preferably comprises a plurality of coating additives (iii.1) in a total amount by weight comprised in the range 0.1% to 30% relative to the total weight of the (iii) coating matrix, preferably 1% to 20%, more preferably 5% to 10%.

The composition of the present invention can comprise, in addition to the (i) mixture and (iii) coating matrix, also (ii) at least one pharmaceutical or food grade additive and/or excipient, i.e. a substance with no therapeutic activity suitable for pharmaceutical or food use. In the context of the present invention the ingredients acceptable for pharmaceutical or food use comprise all the auxiliary substances known to the person skilled in the art, such as, by way of non-limiting example, diluents, solvents (including water, glycerine, ethyl alcohol), solubilising agents, thickeners, sweeteners, flavourings, colourants, lubricants, surfactants, antimicrobials, antioxidants, preservatives, buffers for stabilising the pH and mixtures thereof. Non-limiting examples of such substances are maltodextrins, phosphate buffers, bases such as sodium hydroxide, xanthan gum, guar gum, fructose, and natural or artificial flavourings.

The subject matter of the present invention further relates to a pharmaceutical composition, nutraceutical composition, dietary supplement product or a food product or a food for special medical purposes, a feed, a feed additive or a composition for a medical device comprising or, alternatively, consisting of the composition of the present invention, comprising (i), (iii) and, optionally, (ii) and/or (iii.1) according to any one of the embodiments of the invention.

In the context of the present invention, the term “medical device” is used within the meaning according to Italian Legislative Decree no. 46 of 24 Feb. 1997, or according to the new Medical Device Regulation (EU) 2017/745 (MDR).

In a preferred embodiment of said pharmaceutical composition, nutraceutical composition, dietary supplement product or food product or a food for special medical purposes, feed, feed additive or a composition for a medical device which comprises the composition of the invention, comprising said (i) and (iii) and, optionally, (ii) and/or (iii.1), the (i) mixture comprises or, alternatively, consists of leucine or a mixture of leucine, isoleucine and valine or a whey protein and, optionally, at least one amino acid selected in the aforesaid group A or group B.

The composition of the present invention can be, by way of non-limiting example, in a liquid form, as a solution, two-phase liquid system, suspension or syrup, in semisolid form, as a gel, cream or foam, or in solid form, as a powder, granules, flakes, aggregates, capsules, tablets, bars and equivalent forms.

The composition of the invention is preferably for oral (enteral) use, preferably in solid form as granules, microgranules, flakes or powder; even more preferably in a pharmaceutical form as a tablet, capsule or soft-gel capsule; for example in the form of microcapsules or microgranules to be inserted in capsules to be swallowed, to be inserted in supplements for humans and animals or to be inserted into complete foods for humans and animals. Alternatively, the composition of the invention is for oral use in liquid form as a suspension, for example granules, microgranules or powder in suspension.

When the composition of the invention is in tablet form it means that the aggregate which forms between the active components comprised in the (i) mixture (i.e. (a) and/or (b) and, optionally, (c)) and the (iii) lipid matrix that embeds or incorporates said active components is processed so as to form a tablet.

The composition of the invention in tablet form is not a tablet coated with the (iii) lipid matrix of the invention.

Unless specified otherwise, the indication that a composition or mixture “comprises” one or more components or substances means that other components or substances can be present in addition to the one or ones specifically indicated.

The subject matter of the present invention relates to the composition of the invention, comprising (i) and (iii) and, optionally, (ii) and/or (iii.1) according to any one of the embodiments of the invention, obtained/obtainable according to the above-described process of preparation of the present invention (steps (I), (II) and (III)).

The subject matter of the present invention relates to the composition of the invention comprising said (i) and (iii) and, optionally, (ii) and/or (iii.1) according to any one of the embodiments of the invention, as a pharmaceutical composition, nutraceutical composition, dietary supplement product or a food product or a food for special medical purposes, a feed, a feed additive or a composition for a medical device, for use as a medicament.

The subject matter of the present invention further relates to the composition of the invention comprising said (i) and (iii) and, optionally, (ii) and/or (iii.1) according to any one of the embodiments of the invention, as a pharmaceutical composition, nutraceutical composition, dietary supplement product or a food product or a food for special medical purposes, a feed, a feed additive or a composition for a medical device, for use in a method of treatment with the supply of amino acids to a monogastric subject, preferably a human subject or pig.

The term “supply of amino acids” means the average daily supply of amino acids (or proteins or analogues thereof) for the subject's normal development or a greater or more rapid development compared to the average development of the species to which the subject belongs.

The subject matter of the present invention further relates to the composition of the invention comprising said (i) and (iii) and, optionally, (ii) and/or (iii.1) according to the above-described embodiments for use in a method of preventive and/or curative treatment of a protein deficiency and of a pathology, symptom and/or disorder associated with said protein deficiency, in a subject in a state of need.

A mild protein deficiency can cause: a reduction in metabolic efficiency (for example, easy bleeding, slow wound healing, etc.), reduction in the corpuscular elements in the blood, weight loss (as an effect of muscle reduction, reduction in muscle volumes, premature fatigue, difficulty in concentrating and learning difficulties, moodiness, muscle and/or joint and/or bone pain, variations in glycaemia, and greater susceptibility to infections.

Less frequently, a mild protein deficiency can also cause: anxiety (due to the altered synthesis of neurotransmitters), a reduction in athletic performance (reduced compensation of the training stimulus), sleep alterations (some hypothesise that this may be caused by the alteration in the synthesis of tryptophan and serotonin) and digestive imbalances (proteins enable the natural synthesis of digestive enzymes).

Furthermore, a protein deficiency can generate more severe symptoms or disorders or pathologies, such as muscle wasting (consisting in self-digestion of muscle proteins to produce energy), a reduction in muscle mass and strength and a severe reduction in all the protein-based components, such as nails, hair, skin, enzymes, neurotransmitters, hormones, and immunoglobulins.

In one embodiment, the composition of the invention is for use in a method of preventive and/or curative treatment of a reduction in muscle mass and/or reduction in muscular strength and of a pathology, symptom and/or disorder associated with said reduction in muscle mass and/or reduction in muscular strength, such as, for example, sarcopenia, muscle atrophy, muscular dystrophy or muscle catabolism, in a subject in a state of need.

The subject matter of the present description further relates to a method for the preventive and/or curative treatment with a supply of amino acids or of a protein deficiency and of pathologies, symptoms and/or disorders associated with said protein deficiency, wherein said treatment comprises the administration of the composition of the invention as defined above in a subject in need; in particular for the preventive and/or curative treatment of a reduction in muscle mass and/or muscular strength.

In the context of the present invention “method of treatment” means an intervention comprising the administration of a substance or mixture of substances or combination of the same and having the aim of eliminating, reducing/decreasing or preventing a pathology or disease and the symptoms or disorders associated therewith.

Finally, the subject matter of the present invention relates to the non-therapeutic use of the composition of the invention according to the above-described embodiments, wherein said use is for non-therapeutic treatment with a supply of amino acids or of a protein deficiency and of a disorder associated with said protein deficiency in a subject in a state of need, wherein said non-therapeutic use comprises the administration of said composition; said disorder associated with said protein deficiency is preferably a reduction in muscle mass and/or muscular strength.

Finally, the subject matter of the present invention relates to the use of the composition of the invention, comprising (i) and (iii) and, optionally, (ii) and/or (iii.1) according to any one of the embodiments of the invention, for preparing a feed or an additive for feeds for monogastric animals, preferably pigs.

Unless specified otherwise, the expression “the composition or mixture comprises a component in an amount comprised in an range x to y” means that said component can be present in the composition or mixture in all the amounts present in said range, even if not explicitly stated, the endpoints of the range included.

Embodiments (FRn) of the present invention are disclosed below:

FR1. A method for preparing a composition comprising at least one amino acid, preferably at least two amino acids or acceptable pharmaceutical or food grade salts thereof, said method comprising the steps of:
evaluating and/or quantifying, for a human subject or an animal, preferably a monogastric animal, at least a first parameter selected from the group comprising or, alternatively, consisting of: gender, age, race or breed or amino acid composition of the muscle fibre on the basis of the race or breed, type of sports activity engaged in, type of work engaged in, daily diet or a set of two or more of said first parameters; and/or
evaluating and/or quantifying, for a human subject or an animal, preferably a monogastric animal, at least a second parameter selected from the group comprising or, alternatively, consisting of: plasma nitrogen concentration, blood creatinine, blood creatine phosphokinase, blood lactic acid or lactate after physical exertion, number of daily steps, or saliva sample or parameters obtained from a genetic characterisation or a set of said second parameters; followed by,
selecting, on the basis of said at least a first parameter or set of said first parameters and on the basis of said at least a second parameter or set of said second parameters, at least one amino acid, preferably at least two amino acids; followed by
selecting, on the basis of said at least a first parameter or set of said first parameters and on the basis of said at least a second parameter or set of said second parameters, a first amount of said at least one amino acid, preferably of said at least two amino acids; followed by,
formulating said at least one amino acid, preferably said at least two amino acids, selected and quantified in the preceding steps in a composition suitable for being administered to said subject or animal.

FR2. The method according to FR1, wherein said first and/or second parameter is evaluated and quantified by analysing the amino acid composition of the muscle fibre and/or through analysis of saliva samples or blood samples or blood tests on the subject or animal; it is preferably determined by collecting a saliva sample to collect the DNA of the single individual and/or by determining the presence of genes involved in the anabolic process and/or correlatable to the composition of the muscle fibre and/or correlatable to specific pathologies by carrying out standard methods.

FR3. The method according to FR1 or FR2, wherein the steps of measuring said first parameter and of evaluating said second parameter are further followed by a step of:
selecting, on the basis of said at least a first parameter or set of said first parameters and/or on the basis of said at least a second parameter or set of said second parameters, at least one non-amino acid ingredient selected from among one or more organic or inorganic acids, one or more aromatic components, at least one vitamin, a mineral salt, an antioxidant, a vegetable extract, a probiotic bacterial strain and mixtures thereof; followed by,
selecting, on the basis of said at least a first parameter or set of said first parameters and on the basis of said at least a second parameter or set of said second parameters, a second amount of said at least one non-amino acid ingredient; followed by,
formulating said at least one non-amino acid ingredient in said second amount together with said at least one amino acid, preferably said at least two amino acids or salts thereof in said composition.

FR4. A composition obtained by means of the method according to any one of FR1-FR3 comprising:
(i) a mixture which comprises or, alternatively, consists of at least one amino acid, preferably two amino acids or acceptable pharmaceutical or food grade salts thereof, and, optionally,
(ii) at least one acceptable pharmaceutical or food grade additive and/or excipient.

FR5. The composition according to FR4, wherein at least one of said at least one amino acid, preferably said at least two amino acids or salts thereof, is an essential amino acid selected from the group consisting of: histidine, isoleucine, leucine, lysine, methionine, phenylalanine, threonine, tryptophan and valine; preferably phenylalanine, lysine, methionine, threonine, tryptophan and leucine, more preferably leucine.

FR6. The composition according to FR4 or FR5, wherein said composition further comprises:
(iii) a coating matrix comprising or, alternatively, consisting of at least one saturated or unsaturated fatty acid having a carbon number comprised in the range C10-C30, preferably C14-C24, and/or at least one triglyceride having chains of saturated or unsaturated fatty acids, having a carbon number comprised in the range C6-C30, preferably C14-C24, and/or waxes having a number of carbon atoms comprised in the range C16-C36, preferably C24-C36; wherein said (iii) coating matrix incorporates and/or microencapsulates said (i) mixture, and wherein said (iii) coating matrix enables a controlled release of the amino acid after administration to said subject, thus assuring a larger amount and constancy of amino acids in the blood and a more constant blood bioavailability of amino acids over a 24 hour period by limiting the fluctuations thereof between the main meals.

FR7. The composition according to any one of embodiments FR4 to FR6, wherein said (iii) coating matrix comprising or, alternatively, consisting of: at least one hydrogenated fatty acid of vegetable and/or animal origin, preferably of vegetable origin; and/or at least one triglyceride hydrogenated or non-hydrogenated of vegetable and/or animal origin; the triglycerides of animal origin are preferably selected from among: chicken fat, hydrogenated chicken fat, beef tallow and pork lard; and/or a wax, preferably a beeswax.

FR8. The composition according to any one of embodiments FR4 to FR7, wherein said composition is for use in a method of preventive and/or curative treatment of a protein deficiency and of a pathology, symptom and/or disorder associated with said protein deficiency, in a subject in a state of need.

FR9. The composition for use according to any one of embodiments FR4-FR8, wherein said composition is for human or animal use, preferably for a monogastric animal, in a method of preventive and/or curative treatment of a reduction in muscle mass and/or reduction in muscular strength and of a pathology, symptom and/or disorder associated with said reduction in muscle mass and/or muscular strength, in a subject in a state of need.

FR10. The composition for use according to any one of FR4-FR9, wherein said composition is for use in a method of preventive and/or curative treatment of sarcopenia.

FR11. A non-therapeutic use of the composition according to any one of embodiments FR4 to FR7, wherein said use is for the non-therapeutic treatment of a protein deficiency and of a disorder associated with said protein deficiency, wherein said non-therapeutic use comprises the administration of said composition to a subject in a state of need; preferably said disorder associated with said protein deficiency is a reduction in muscle mass and/or muscular strength.

Unless specified otherwise, the expression that the composition comprises a component in an amount “comprised in an range x to y” means that said component can be present in the composition in all the amounts present in said range, even if not explicitly stated, the endpoints of the range included.

### REFERENCES

1. Dideriksen K., Reitelseder S., Holm L. (2013). Influence of amino acids, dietary protein, and physical activity on muscle mass development in humans. Nutrients, 5(3):852-76. doi: 10.3390/nu5030852.

2. Nowson C., O'Connell S. (2015). Protein Requirements and Recommendations for Older People: A Review. Nutrients, 7(8):6874-99. doi: 10.3390/nu7085311.

3. Philips S M (2014). Sports Med, 44:S71-S77. doi 10.1007/s40279-014-0152-3.

4. Dietary Reference Intake (DRI)—2006 Ed.

5. Jager R, Kerksick M C, Campbell B I, et al. (2017). International Society of Sports Nutrition Position Stand: protein and exercise Journal of the International Society of Sports Nutrition (2017) 14:20. DOI 10.1186/s12970-017-0177-8.

6. Mitchell W K, Wilkinson D J, Phillips B E, Lund J N, Smith K, Atherton PJ (2016). Human Skeletal Muscle Protein Metabolism Responses to Amino Acid Nutrition. Adv Nutr, 7(4): 828S-38S. doi:10.3945/an.115.011650.

7. Stokes T, Hector A J, Morton R W, McGlory C, Phillips S M (2018). Recent Perspectives Regarding the Role of Dietary.

8. Elango R, Ball R O, Pencharz P B (2008). Individual amino acid requirements in humans: an update. Current Opinion in Clinical Nutrition and Metabolic Care 2008, 11:34-39.

9. LARN: Livelli of Assunzione of Riferimento of Nutrienti and energia per the popolazione italiana. SICS Editore, 2017.

1. A composition comprising
(i) a mixture of active components which comprises or, alternatively, consists of (a) at least one amino acid, or an acceptable pharmaceutical or food grade salt thereof, and/or (b) a whey protein;
(ii) a controlled-release lipid matrix which embeds or incorporates said (i) mixture of active components, wherein said controlled-release lipid matrix comprises or, alternatively, consists of at least one fatty acid, saturated or unsaturated, free or esterified, having a number of carbon atoms comprised in the range C10-C30, and/or at least one triglyceride having chains of saturated or unsaturated fatty acids, having a carbon number comprised in the range C6-C30, and/or at least one wax having a number of carbon atoms comprised in the range C16-C36; and, optionally, said composition comprises
(ii) at least one acceptable pharmaceutical or food grade additive and/or excipient;
wherein said (iii) lipid matrix enables a gastroprotection and a controlled release in the gut of the active components comprised in said (i) mixture, thus assuring a constant blood bioavailability thereof over a period comprised in the range 2 to 24 hours.

2. The composition according to claim 1, wherein said (a) at least one amino acid is selected in group A comprising or, alternatively, consisting of: histidine, isoleucine, leucine, lysine, methionine, phenylalanine, threonine, tryptophan, valine, arginine, cysteine, tryptophan, glutamine and mixtures thereof.

3. The composition according to claim 1, wherein said (a) at least one amino acid is leucine.

4. The composition according to claim 1, wherein said (a) at least one amino acid is a mixture of leucine, valine and isoleucine.

5. The composition according to claim 1, wherein said (a) at least one amino acid is a mixture of leucine and at least one or more amino acids selected from the group comprising or, alternatively, consisting of: lysine, methionine, threonine, tryptophan, valine, isoleucine, histidine and glutamine.

6. The composition according to claim 1, wherein said (i) mixture of active components further comprises (c) at least one non-amino acid ingredient selected in group C comprising or, alternatively, consisting of: at least one vitamin, preferably a B group vitamin, at least one organic or inorganic acid, at least one mineral salt, preferably an organic or inorganic salt of a cation of Fe, Se, Mg, Ca, K, Zn or Cu, at least one antioxidant, at least one probiotic bacterial strain, at least one prebiotic, at least one enzyme and mixtures thereof.

7. The composition according to claim 1, wherein said (iii) controlled-release lipid matrix is selected from the group comprising or, alternatively consists of: palm oil, sunflower oil, corn oil, rapeseed oil, peanut oil, soybean oil, olive oil, beeswax and mixtures thereof.

8. The composition according to claim 1, wherein said (iii) controlled-release lipid matrix further comprises (iii.1) at least one coating additive selected from the group comprising or, alternatively consisting of: fumed silica, calcium stearate, magnesium stearate, calcium sulphate, precipitated silica, calcium silicate, aluminium silicate and hydrophobic silica.

9. The composition according to claim 1, wherein said composition is a pharmaceutical composition, composition for a medical device, nutraceutical composition, dietary supplement product, food product, food for special medical purposes, feed or feed additive.

10. The composition according to claim 1, wherein said composition is for use as a medicament.

11. The composition according to claim 1, wherein said composition is for use in a method of treatment with a supply of amino acids to a monogastric subject in a state of need, preferably a human subject or a pig.

12. The composition according to claim 1, wherein said composition is for use in a method of treatment of a protein deficiency and of a pathology, symptom and/or disorder associated with said protein deficiency, in a monogastric subject in a state of need, preferably a human subject or a pig.

13. The composition according to claim 1, wherein said composition is for use in a method of preventive and/or curative treatment of a reduction in muscle mass and/or reduction in muscular strength and of a pathology, symptom and/or disorder associated with said reduction in muscle mass and/or muscular strength.

14. The composition according to claim 13, wherein said pathology, symptom and/or disorder associated with said reduction in muscle mass and/or in muscular strength is selected from among sarcopenia, muscle atrophy, muscular dystrophy and muscle catabolism.

15. A method comprising feeding a composition according to claim 1.

16. The method of claim 15, wherein the monogastric animal is a pig.
